(19) United States
(12) Patent Application Publication
(10) Pub. No.: US 2007/0049927 A1
(43) Pub. Date: Mar. 1, 2007

(54) Electrosurgical pencil with a light

(76) Inventor: Darin J. Saltzman, Huntington Beach, CA (US)

Correspondence Address: MANUEL F. DE LA CERRA, 6885 CATAMARAN DRIVE, CARLSBAD, CA, 92011, US

(21) Appl. No.: 11/216,905

(22) Filed: Aug. 31, 2005

(51) Int. Cl. A61B 18/14

(52) U.S. Cl. 606/45

(57) ABSTRACT

An electrosurgical pencil is provided having a light source for illuminating a tissue area of interest. In various embodiments, the light source is a fixed part of the electrosurgical pencil body or the cutting element. The light source may be electrically connected to the power source of the electrosurgical pencil or may have its own independent power source. The light source may also have its own switch or may be electrically coupled to the switch that operates the electromagnetic pencil.

### BACKGROUND OF THE INVENTION

1. Field of the Invention

The field of the present invention relates to electrosurgical pencils.

2. Description of the Related Art

Electrosurgical pencils are well known in the art. For example, U.S. Pat. Nos. 6,361,532 and 6,616,658, hereby incorporated by reference herein in their entirety, including any drawings, generally describe electrosurgical pencils, including manufacturing techniques and circuitry for creating an electrosurgical pencil.

In electrosurgical techniques using electrosurgical pencils, an electrosurgical power source, such as a generator, current from a wall socket, batteries, etc., generates electrical current upon demand, and the current is carried to an electrosurgical pencil having a relatively small blade. In some electrosurgical pencils, the current is transmitted through the blade to the patient and back to the power source through a ground plate attached to the patient. Since the blade is small relative to the ground plate, the energy transferred to the patient through the small blade is concentrated such that a high frequency AC current causes tissue destruction. Typically, such devices have two modes of operation, cutting and coagulation, which are performed with current having different wave forms.

In hand-controlled electrosurgical pencils, an internal switch, usually mounted to a printed circuit board, is incorporated into the pencil housing. The housing additionally includes appropriate buttons to select the desired mode of operation through the printed circuit board (i.e., either a cutting or coagulation signal). In some electrosurgical pencils, an actuation switch is positioned remote from the pencil housing to be controlled by another manner such as by the operator's foot.

It is desirable, therefore, to have an electrosurgical pencil with a light to illuminate a field where the electrosurgical pencil is to be used, in accordance with the present invention to satisfy a long unmet need in the art.

### SUMMARY OF THE INVENTION

The system, method, and devices of the invention each have several aspects, no single one of which is solely responsible for its desirable attributes. Without limiting the scope of this invention, its more prominent features will now be discussed briefly. After considering this discussion, and particularly after reading the section entitled “Detailed Description of Certain Embodiments” one will understand how the features of this invention provide advantages over other electrosurgical pencils.

Disclosed herein is an electrosurgical pencil, comprising: an elongated body comprising a distal end and a proximal end; a connector formed near the distal end of the elongated body configured to receive a cutting element; a connector formed near the proximal end of the elongated body configured to connect to an electrical power source and provide power to the cutting element; and a light source formed on the elongated body.

Also disclosed herein is an electrosurgical pencil, comprising: an elongated body comprising a distal end and a proximal end; a cutting element near the distal end; a connector near the proximal end configured to connect to an electrical power source and provide power to the cutting element; a light source formed on the elongated body, wherein the light source is positioned on the cutting element.

Further disclosed herein is a method of manufacturing an electrosurgical pencil, comprising: forming an elongated body comprising a distal end and a proximal end; forming a connector near the distal end configured to receive a cutting element; forming a connector near the proximal end configured to connect to an electrical power source and provide power to the cutting element; and providing a light source formed on the elongated body.

In addition, disclosed herein is an electrosurgical pencil, comprising: an elongated body comprising a distal end and a proximal end; a connector formed near the distal end of the elongated body configured to receive a cutting element; a connector formed near the proximal end of the elongated body configured to connect to an electrical power source and provide power to the cutting element; and means for providing illumination.

### BRIEF DESCRIPTION OF THE DRAWINGS

FIG. 1 is a perspective view of an electrosurgical pencil having a light source in accordance with one embodiment of the invention.

FIG. 2 is another perspective view of an electrosurgical pencil having a light source in accordance with another embodiment of the invention.

FIG. 3 is a perspective view of an electrosurgical pencil showing various embodiments for switches operating the light source.

FIG. 4 is a perspective view of an electrosurgical pencil having a light source at the end of an extension apparatus.

### DETAILED DESCRIPTION OF THE PREFERRED EMBODIMENT

The following detailed description is directed to certain specific embodiments of the invention. However, the invention can be embodied in a multitude of different ways. In this description, reference is made to the drawings wherein like parts are designated with like numerals throughout.

Adequate lighting of an operation site is of great importance for any surgical procedure. Currently, many such procedures, including, but not limited to, incision, suturing, clamping, cauterizing, irrigating, and various forms of tissue manipulation within a patient's body, are often performed under overhead lighting of high intensity. In particular circumstances, this is not adequate, and surgeons sometimes utilize a light mounted to a harness worn on the head and intended to direct light forwards to facilitate viewing of the operation site where the surgeon is applying surgical instruments. In other circumstances, surgeons require the help of an assistant to direct light from a light source, such as the overhead light or a hand held lighting device, into the sight of the operation. This creates difficulties because the surgeon will have to constantly communicate with the assistant to place the light in a proper position, which causes the surgeon's attention to be diverted from the delicate task of surgery at hand.

In certain kinds of surgery using an electrosurgical pencil, the surgeon has to operate in a deep crevice or hole within a patient's body, and neither overhead lighting nor a head-mounted light is satisfactory. It is desirable, therefore, to have an electrosurgical pencil with a light to illuminate a field where the electrosurgical pencil is to be used, in accordance with the present invention.

Thus, in the first aspect, disclosed herein is an electrosurgical pencil comprising a light source, which light source is configured to illuminate the site of surgery.

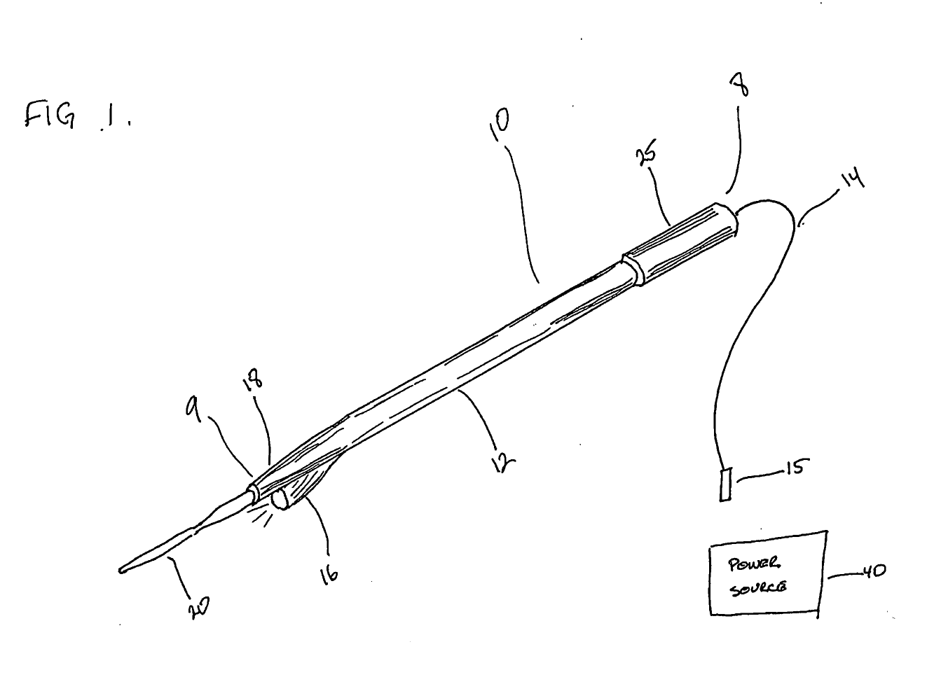

FIG. 1 is a perspective view of an electrosurgical pencil 10 having a light source 16 in accordance with an embodiment of the invention. As referred to herein throughout the Specification, the light source 16 provides illumination in accordance with embodiments of the present invention. Moreover, as referred to herein, the terms electrosurgical pencil and light source as to be given their broadest ordinary meaning. An electrosurgical pencil refers to, but is not limited by, an electric powered surgical scalpel that uses electrical current passed through a cutting element as part of its cutting mechanism. Exemplary examples of an electrosurgical pencil include a Bovie™ pencil. The term “light source” refers to, but is not limited by, a device or object that produces electromagnetic radiation, including ultra-violet light and visible light. The light source also refers to, but is not limited by, an infinite light, point light, and spotlight.

The electrosurgical pencil 10 has an elongated body 12 having a proximal end 8 and a distal end 9. Unless otherwise specified, throughout the present disclosure, the term “proximal” refers to the portion of the electrosurgical pencil, or components thereof, closer to the proximal end 8 than to the distal end 9. Similarly, the term “distal” refers to the portion of the electrosurgical pencil, or components thereof, closer to the distal end 9 than to the proximal end 8.

The elongated body 12 joins a connector portion 25 at the proximal end 8 with an active tip 18 at the distal end 9. Active tip 18 may include a socket for receiving interchangeable cutting elements 20, which serve as an electrode for a circuit formed by the surgical apparatus.

The connector portion 25 may be connected to a cable 14 leading from a plug 15. The plug 15 is configured to be inserted into a power source 40 to provide power to the electrosurgical pencil 10 and the light source 16. In electrosurgery, electrical current flows through a circuit that begins at the power source 40, goes through the cable 14 to the cutting element 20, and then returns from the patient by way of a dispersive electrode (not shown) to the power source 40.

In some embodiments, the power source is a signal generator, which generates the current at the appropriate frequency and level. In some embodiments, the electrosurgical pencil is directly plugged into a wall socket. Alternatively, the electrosurgical pencil may be electrically connected to a separate device, which device is in turn plugged into a wall socket. In other embodiments, the power source is at least one battery that provides power to the electrosurgical pencil. The battery may be housed within the electrosurgical pencil or it may be housed in a device that is electrically connected to the electrosurgical pencil.

The relatively small contact area between the cutting element 20 and tissue being operated on causes a high current density that heats the tissue at this point, thus allowing for the “electrosurgical” cutting of tissue. Because of the delicate nature of many electrosurgical procedures, it is important to have an illuminated area where the electrosurgical pencil is used. Thus, with the present embodiment, a light source 16 provides illumination to the tissue area being operated on.

In the embodiment depicted in FIG. 1, the light source 16 is part of the elongated body 12 positioned between the proximal end 8 and the distal end 9 of the electrosurgical pencil 10. An aperture is formed in the elongated body 12 where a housing for the light source 16 is placed and connected to the elongated body 12. Electronic connectors are provided through the light source 16 housing into the elongated body 12 where the electrical connectors form an electrical circuit to provide power to the light source 16. In one embodiment, this electrical circuit is connected to the same power source that provides power to the cutting element 20. In another embodiment, the light source 16 is on a separate electrical circuit than the one providing power to the cutting element 20.

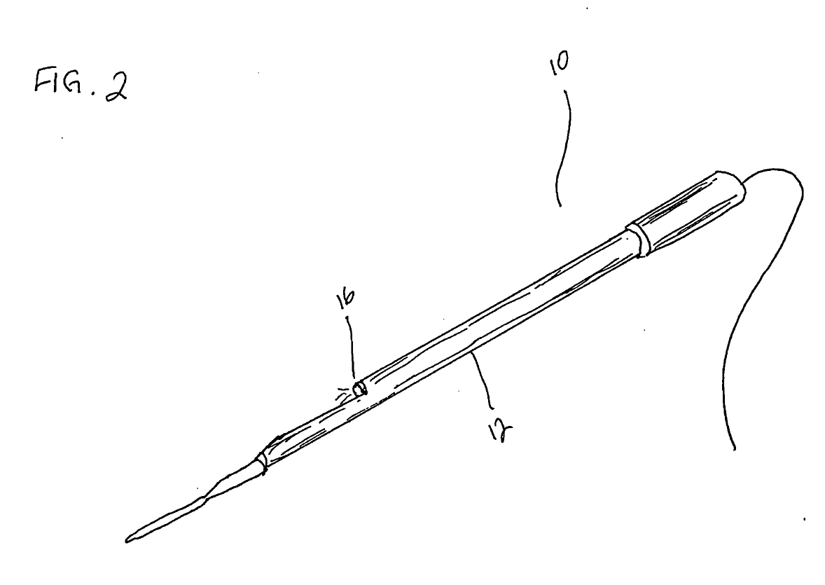

The light source 16 may be positioned in various different places on the electrosurgical pencil 10. For example, FIG. 2 is a side view of an electrosurgical pencil 10 having the light source 16 within the elongated body 20.

In one embodiment, the light source 16 may be located at the distal end 9 of the elongated body 12. An aperture may be formed at the distal end 9 of the elongated body 12 and the light source 16 may be formed within the aperture. Light from the light source 16 would then exit the housing from the distal end 9 providing illumination of the cutting element 20 and the tissue being operated on.

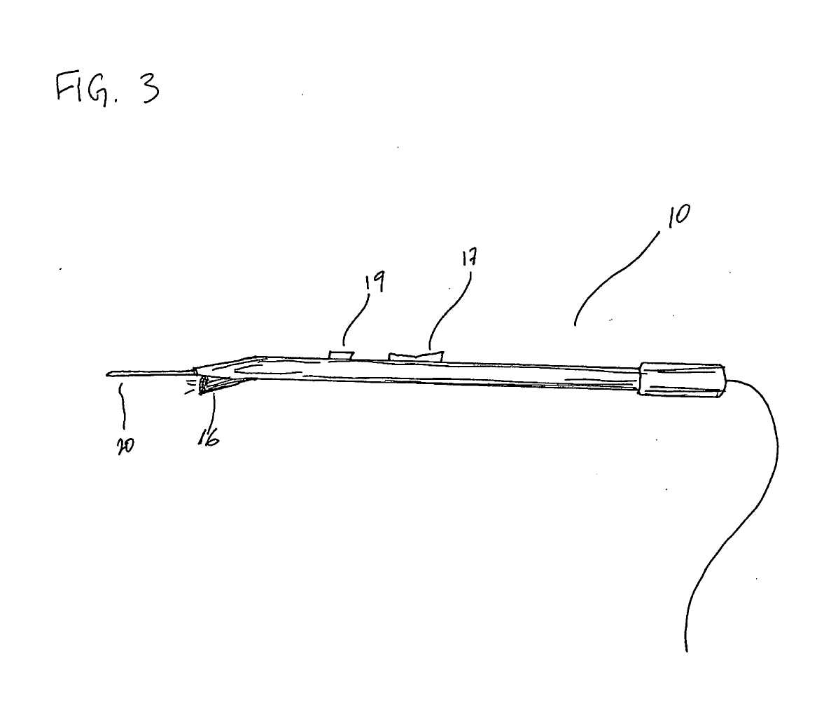

FIG. 3 also illustrates various options for providing switching elements to operate the electrosurgical pencil 10 and the light source 16. The electrosurgical pencil 10 may incorporate switch 17 that initiates current flow though the cutting element 20. This switch 17 may be a rocker switch as depicted in FIG. 3 with two modes, including a mode to provide sufficient current to cut tissue with the active distal end 20 and a mode to provide sufficient current to coagulate blood but not cut tissue. In one embodiment, the switch 17 may also activate the light source 16, such that when the switch 17 is activated to provide current to the active distal end 20, the electronic circuitry of the electrosurgical pencil 10 also provides power to the light source 16, thereby providing illumination.

In another embodiment, a light source switch 19 may be provided near the distal end of the electrosurgical pencil 10. The light source switch 19 may be connected to electronic circuitry within the electrosurgical pencil 10, where the light source switch 19 may be used to provide power to the light source 19 independent of whether the switch 17 is activated and providing power to the cutting element 20.

In some embodiments, the light source 16 and the cutting element 20 are controlled by the same switch. In these embodiments, the light source 16 is illuminated whenever the surgeon uses the switch to activate the cutting element 20. In some of these embodiments, the light source 16 and the cutting element 20 are on the same electrical circuit, while in other embodiments the light source 16 and the cutting element 20 are on different electrical circuit, even thought they are being controlled by the same switch.

In other embodiments, the light source 16 is connected to a separate switch. In these embodiments, the surgeon can turn the light source 16 on or off independently of the cutting element 20. Thus, the surgeon can leave the light on and the cutting element off while the surgeon is contemplating the next incision, or when the surgeon is exploring the area to be operated on.

In further embodiments, the light source 16 comprises at least two light sources of different intensities. Thus, for example, the surgeon can turn on a light of lower intensity and then turn on a light of higher intensity when the cutting element 20 is also activated. In this embodiment, the surgeon can explore the area of operation with light of lower intensity and then provide greater illumination during the actual operation.

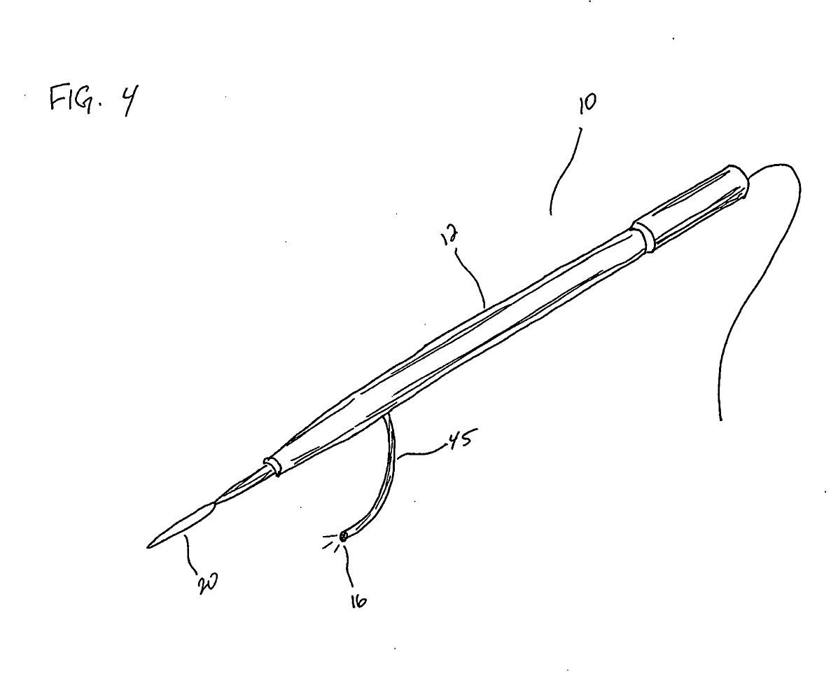

Referring to FIG. 4, in other embodiments, the light source 16 may be used in conjunction with an extension apparatus 45 where the light source 16 is able to be extended from the electrosurgical pencil 10 via a rigid or flexible connector 45 that keeps the light source 16 in electrical communication with the electrosurgical pencil 10. In one embodiment using the extension apparatus 45, the light source 16 could be mounted to tissue inside the patient, with the surgeon having more freedom to move the light source 16 with some independence from the electrosurgical pencil 10, thereby adjusting the direction and/or focus of the lighting. The extension apparatus 45 may be a flexible cable with the light source connected to one end of the extension apparatus 45 and the electrosurgical pencil 10 connected to the other end of the extension apparatus 45. In this embodiment, the surgeon has more freedom to move the light source 16 with some independence from the electrosurgical pencil 10 by flexing the cable of the extension apparatus 45 to illuminate a tissue area of interest.

In various embodiments, the light source 16 is configured to provide lighting of a selected frequency range and/or with an adjustable focus ranging from substantially diffuse to a tightly focused beam. The desired light in the light source 16 is preferably generated by a light-emitting diode (LED). Although any type of appropriate light may be used in the light source 16, such as a conventional incandescent lamp, an LED is preferable for its small size, higher efficiency, and lower operating temperature and heat release. It is noteworthy that the problem of collateral heat release from an operating LED has been addressed in U.S. Pat. No. 6,028,694 to Schmidt titled “Illumination Device Using Pulse Width Modulation of a LED,” which is incorporated herein by reference. A suitable LED for use in the present invention is BP280CW4K-3.6Vf-050T, manufactured by LEDtronic™, of Torrance, Calif., USA.

It is noted that in surgery it is very important for the surgeon to quickly see colors accurately. To an experienced surgeon, the hue and intensity of even a single basic color can convey significant information about the condition of particular tissues and hence the patient. White light is generally considered best for surgical applications but other colors, perhaps even ultra-violet light, may reveal details that might otherwise be easily missed during difficult viewing in a confined space. By careful choice of the LED (or an adequate substitute) users can readily adapt the present invention to suit their specific needs. In some embodiments, the device of the present invention is fitted with a light of a single color. In other embodiments, the device of the present invention exhibits different color lights. In yet another embodiment, the light on the device of the present invention is interchangeable so that the surgeon can use a light of appropriate color for the task at hand.

In some embodiments, the light source 16 of the electrosurgical pencil 10 has a power source that is independent of the electrical circuitry of the cutting element 20. For example, the electrosurgical pencil 10 may be configured with a battery power supply within the elongated body 12 in order to power the light source 16. In another embodiment, the light source 16 may be configured with its own separate electrical circuitry that utilizes power from the power source without drawing power from the current flowing through wire 26. For example, another wire may be connected directly from pin 24 to the light 28 of the light source 16.

While the present invention comprehends the use of a low cost disposable electrosurgical pencil 10 with interchangeable tips, it should be understood that it is within the purview of the invention, as discussed above, to use a reusable electrosurgical pencil 10. Electrosurgical pencils are typically disposable in order to prevent contamination from a patient's blood and because sterilizing conditions necessary for re-usable devices have a tendency to weaken the electronic components of such an arrangement. Such difficulties make it appropriate and desirable to use disposable arrangements in accordance with this invention, although reusable arrangements are also within the scope of this invention.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others.

What is claimed is:

1. An electrosurgical pencil, comprising:
an elongated body comprising a distal end and a proximal end;
a connector on the elongated body configured to receive a cutting element;
a connector configured to connect to an electrical power source and provide power to the cutting element; and
a light source coupled to the elongated body.

2. The electrosurgical pencil of claim 1, further comprising a circuit configured to provide power to the light source, wherein the circuit is located within the elongated body.

3. The electrosurgical pencil of claim 2, wherein the circuit configured to provide power to the light source comprises a battery source, wherein the battery source is located within the elongated body.

4. The electrosurgical pencil of claim 2, further comprising a switch located on the elongated body, wherein the circuit configured to provide power to the light source is in operable communication with the switch.

5. The electrosurgical pencil of claim 4, wherein the switch also controls the cutting element.

6. The electrosurgical pencil of claim 1, wherein the light source comprises an LED.

7. The electrosurgical pencil of claim 1, wherein the light source comprises an incandescent lamp.

8. The electrosurgical pencil of claim 1, wherein the light source is positioned on the elongated body between distal and proximal ends of the elongated body.

9. The electrosurgical pencil of claim 1, wherein the light source is positioned near the distal end of the elongated body.

10. The electrosurgical pencil of claim 1, further comprising an extension apparatus in operable connection with the light source and the elongated body.

11. The electrosurgical pencil of claim 1, wherein the light source has an adjustable focus.

12. The electrosurgical pencil of claim 1, wherein the electrosurgical pencil is disposable.

13. The electrosurgical pencil of claim 1, wherein the electrosurgical pencil is reusable.

14. An electrosurgical pencil, comprising:
a handheld housing;
a cutting element attached to the housing; and
a light source formed on the housing positioned to illuminate an area around the cutting element.

15. The electrosurgical pencil of claim 14, wherein the light source comprises an LED.

16. The electrosurgical pencil of claim 14, wherein the light source has an adjustable focus.

17. A method of manufacturing an electrosurgical pencil comprising attaching an integral light source to a housing that also supports an electrosurgical cutting instrument.

18. The method of claim 17, wherein the housing forms an elongated body and the light source is positioned on the elongated body between the distal and proximal ends of the elongated body.

19. The method of claim 17, wherein the light source is positioned near the distal end of the elongated body.

20. An electrosurgical pencil manufactured in accordance with the method of claim 17.

21. An electrosurgical pencil, comprising:
an elongated body comprising a distal end and a proximal end;
a connector formed near the distal end of the elongated body configured to receive a cutting element;
means for providing illumination of a region around the distal end of the elongated body.

22. The electrosurgical pencil of claim 21, further comprising a circuit configured to provide power to said means for providing illumination, wherein the circuit is located within the elongated body.
